(19) United States
(12) Patent Application Publication
(10) Pub. No.: US 2005/0043789 A1
(43) Pub. Date: Feb. 24, 2005

(54) Anti-thrombogenic coatings for biomedical devices

(76) Inventors: Christopher W. Widenhouse, Gainesville, FL (US); Eugene P. Goldberg, Mount Dora, FL (US); James M. Seeger, Gainesville, FL (US)

Correspondence Address: MILES & STOCKBRIDGE PC, 1751 PINNACLE DRIVE, SUITE 500, MCLEAN, VA, 22102-3833, US

(21) Appl. No.: 10/936,534

(22) Filed: Sep. 9, 2004

Related U.S. Application Data
(63) Continuation of application No. 09/950,821, filed on Sep. 13, 2001, now abandoned.
Application No. 09/950,821 is a continuation-in-part of application No. 09/113,375, filed on Jul. 10, 1998, now abandoned.
(60) Provisional application No. 60/052,150, filed on Jul. 10, 1997.

(51) Int. Cl. B05D 3/00; A61F 2/06

(52) U.S. Cl. 623/1.46; 427/2.1; 623/921

(57) ABSTRACT

A device comprising a substrate having a surface for contacting blood wherein the surface has a continuum of thrombus-resistant polymeric material coated on and extending into the substrate to anchor the coating thereon. The device may be prepared by a method comprising: (a) contacting the substrate with a solution of monomer or oligomer in a solvent to wet the surface and impregnate the substrate below the surface to a substantial depth; (b) removing the solvent to form a continuum of the monomer or oligomer on the surface and within the substrate to a substantial depth; and (c) polymerizing the monomer or oligomer to form a substantial continuum of polymer coating on the surface and extending to a substantial depth in the substrate, thereby firmly anchoring the polymer coating on the surface.

### BACKGROUND OF THE INVENTION

1. Related Application

Reference is hereby made to provisional patent application Ser. No. 60/052,150 filed Jul. 10, 1997, the benefit of the filing date of which is claimed herein.

2. Field of the Invention

Vascular prostheses or grafts are conventionally constructed of expanded polytetrafluoroethylene (ePTFE), e.g., GORE-TEX® or woven, knitted or braided polyethylene terephthalate (PET) fabrics [Greisler, “New Biologic and Synthetic Vascular Prostheses,” Chapter 2, R. G. Landes Co., Austin, Tex. (1991)]. Vascular replacements formed from these materials have found widespread clinical use. However, there are serious limitations on their use, especially for small diameters, inasmuch as they may occlude within 24 months after implantation, up to 50% or more of their internal diameter when blood flows therethrough due to thrombogenicity [Underwood et al, International J. Artificial Organs, Vol. 11(4), pages 272-276 (1988)].

Synthetic small diameter vascular prostheses or grafts are defined as those having an internal diameter of 6 mm or less. The small diameter grafts are useful for peripheral repairs of extremities and small arteries such as the coronary arteries. They have been found to substantially occlude in less than 5-10 years [Pevec et al, “Femoropopliteal reconstruction with knitted, nonvelour Dacron versus expanded polytetrafluoroethylene,” J. Vasc. Surg., Vol. 16(1), pages 60-65 (1992)]. Smaller diameters (less than 3-4 mm) are more problematic and likely to occlude in less than 2-3 years. Therefore, a successful small diameter vascular prosthesis (SDVP) for arterial replacement does not exist.

Silicones, in particular, polydialkylsilicones such as polydimethylsiloxane (PDMS), are low surface energy, hydrophobic polymers which resist adhesion of many materials from aqueous environments. Silicone rubber has been found to have a higher affinity for albumin than fibrinogen in competitive adsorption studies, indicating that the material has a low thrombogenicity [Cooper et al, “A comparison of the adsorption of three adhesive proteins to biomaterial surfaces,” J. Biomaterial Sci. Polymer Edn., Vol. 3(1), pages 27-47 (1991)]. Silastic® and silica-free PDMS have significantly lower platelet cell adhesion than polyethylene [Ip et al, “Platelet consumption by NHLBI reference materials and silastic,” J. Biomedical Materials Res., Vol. 25, pages 1321-1324 (1991)]. Silicone-coated PET has reduced thrombogenicity [Norgren et al, “Experimental evaluation of polymerized dacron grafts in the iliac position of pigs,” Annals of Vascular Surgery, Vol. 4, pages 575-579 (1990)] and reduced inflammatory reactions [Granke et al, “Analysis of graft healing in a new elastomer-coated vascular prosthesis,” Cardiovascular Surgery, Vol. 1(3), pages 254-261 (1993)]. A prosthesis made entirely of silicone which allowed tissue in-growth remained 86% patent in canines after 8 weeks [Whalen et al, “A new, all silicone rubber small vessel prosthesis,” ASAIO Journal, Vol. 38, pages M207-M212 (1992)].

U.S. Pat. No. 4,687,482 to Hanson discloses an attempt to coat conventional vascular prosthesis materials with silicone to decrease their thrombogenicity. Hanson demonstrates reduced platelet consumption and reduced thrombogenicity with a PDMS-coated prosthesis [Hanson et al, “Vascular thrombus formation,” Annals NY Acad. Sci., Vol. 516, pages 653-661 (1987)]. More specifically, Hanson discloses exposing vascular prostheses to hexane solvent solutions of various silicones. Hexane, however, will only swell PET, for example, about 2% over a 24-hour period. Simply allowing hexane solutions of silicones to flow over the graft material would not be sufficient to result in diffusion into the substrate of significant amounts of silicone. Thus, there is virtually no significant bonding of the silicone coating to the substrate. Studies have shown that the Hanson-type prosthesis is subject to delamination of the silicone coating from the prosthesis substrate upon mechanical flexing, especially upon suturing. No significant clinical use for such silicone coated devices has, therefore, occurred. Other vascular prostheses and devices having surfaces for contacting blood are described in U.S. Pat. Nos. 3,974,526; 4,906,465 and 5,192,308. These devices, however, have found only limited use.

It is an object of the present invention to provide a novel method for providing strongly adherent, thrombus-resistant, polymeric coatings on materials conventionally employed in constructing vascular prostheses and other devices having surfaces for contacting blood, as well as the devices themselves.

### SUMMARY OF THE INVENTION

The above and other objects are realized by the present invention, one embodiment of which relates to a method of fabricating a device comprising a substrate having a surface of low thrombogenicity for contact with blood comprising the steps of:
(a) contacting the substrate with a solution of monomer or oligomer in a solvent to wet the surface and impregnate the substrate below the surface thereof to a substantial depth with the solution;
(b) removing the solvent to form a substantial continuum of the monomer or oligomer on the surface and within the substrate to that substantial depth; and
(c) polymerizing the monomer or oligomer to form a substantial continuum of polymer coating on the surface and extending to that substantial depth in the substrate, thereby firmly anchoring the polymer coating on the surface; it being noted that the order of steps (b) and (c) may be reversed.

A further embodiment of the invention concerns the device produced by the above-described method.

Another embodiment of the invention comprises a device with a substrate having a surface for contacting blood, the surface comprising a substantial continuum of thrombus-resistant polymeric material coated on and extending into the substrate to a substantial depth.

### BRIEF DESCRIPTION OF THE DRAWINGS

FIG. 1 is a cls spectra comparison.

FIGS. 2 and 3 are schematic depictions of testing apparatus.

FIGS. 4, 5 and 6 are SEM micrographs.

FIG. 7 is a schematic representation of a vascular prosthesis according to the invention.

### DETAILED DESCRIPTION OF THE INVENTION

The novel method of the invention essentially comprises a swelling or impregnation of the substrate intended for contacting blood with a monomer or reactive oligomer in an organic solvent, e.g., a polydimethylsiloxane precursor in methylene chloride, chloroform or hexane, whereby the polymer precursor is diffused throughout a substantial depth of the substrate, entrapping molecules of the precursor therewithin and coating the surface thereof. Upon de-swelling or removal of the solvent and subsequent or concomitant solvent removal and reaction, the polymerized siloxane remains bound within and upon the substrate, thereby providing a stable surface coating anchored to the substrate by the continuum of polymer (e.g., polysiloxane) extending into the substrate. The techniques utilized heretofore in the prior art such as in U.S. Pat. No. 4,687,482 (Hanson) applied a surface coating which was inadequately adherent to the substrate.

The invention will be described hereinbelow with particular reference to forming thrombus-resistant polysiloxane coatings on the luminal surfaces of vascular prostheses. It will be understood by those skilled in the art, however, that the invention is equally applicable to and, indeed, embraces forming such coatings on any device having a substrate designed for contacting blood such as, e.g., catheters, artificial hearts, ventricular grafts, cardiovascular sutures, ventricular assist devices, intraaortic balloon pumps, pulmonary artery catheters, ventricular patches, metabolic support catheters, pacer leads and the like.

The invention will also be described hereinbelow with particular reference to coatings formed from dimethylsiloxane monomers and oligomers. It will be equally understood by those skilled in the art, however, that the invention is equally applicable to and, indeed, also embraces forming such coatings from other monomers and/or oligomers which form thrombus-resistant polymeric coatings, such as, e.g., fluorosiloxanes, phenylsiloxanes, hydrophilic alkylene oxide siloxanes, other alkyl siloxanes, as well as copolymers and/or mixtures thereof.

It will be understood by those skilled in the art that the monomer/oligomer system may comprise a two-part cure system such as the Shin-Etsu two-part system of the appended examples (a platinum catalyzed vinyl addition-type reaction system), as well as room temperature vulcanization (RTV) systems, other catalyst activated systems, including platinum, peroxide or UV, photo or radiation initiator systems, and condensation-type silicone systems.

The substrate may comprise any foraminous or porous or otherwise impregnable material suitable for contact with blood. Although it is preferred to employ expanded polymer substrates such as polytetrafluoroethylene (e.g., GORE-TEX®) or woven, knitted or braided fabrics constructed of polyester such as polyethylene terephthalate (e.g., Dacron®), it will be understood by those skilled in the art that the devices of the invention may be constructed of any suitable material capable of being surface-coated and impregnated to a substantial depth to the substrate below the surface with the solution of monomer or oligomer. Among such materials are, for example, fibers, tubes, molded parts, woven and non-woven fabrics, expanded or knitted, braided or woven substrates or continuous tubular/non-woven/extruded structure/conduits formed of other polyesters, polyamides, polysiloxanes, epoxy resins, polycarbonates, polyurethanes, polyolefins (e.g., polypropylene, polyethylene, etc.), polysulfones, polyimides, and compositions which may comprise metal, glass and ceramic materials and the like having a porous or microporous cross-sectioned structure or a surface capable of being swollen or penetrated by the above-mentioned solutions.

The particular solvent employed, the length of time of contact of the coating system with the substrate, concentration of monomer or oligomer in the solution and other process parameters will depend in each instance upon the nature of the device, the thickness of surface coating desired and other conditions specific to the particular application contemplated. Exposure of the substrate to the coating system may be effected by dipping, spraying, calendaring, surface casting or any other suitable technique known in the art and readily understood by the skilled artisan. It is only necessary to expose the substrate to the coating solution for a length of time sufficient to penetrate the pores and/or interstices of the substrate to the depth desired.

Suitable solvents include those capable of swelling or diffusing into the pores and/or interstices of the substrate to be coated and which, of course, do not deleteriously affect the structure thereof. They should also be capable of easy removal from the coated and impregnated substrate after polymerization of the monomer and/or oligomer, e.g., by thermal evaporation, reduced pressure (vacuum) evaporation or a combination thereof.

The table below sets forth the relevant characteristics for a variety of solvents with respect to this applicability for swelling PET:

TABLE 1
Solubility Parameters And Maximum Swelling For PET
In Various Solvents

| | | | Percent Weight Uptake |
|---|---|---|---|
| | | | by PET in 24 hours |
| Solvent, Monomer | Solubility | | (swelling at room temperature |
| or Solution | Parameter | | unless otherwise noted) |
| Acetone | 9.9 | s | 1% |
| Cyclohexane | 8.2 | p | Less than 1% |
| Dimethylsulfoxide (DMSO) | 12.0 | m | 2% |
| Hexane | 7.3 | p | 2% |
| THF | 9.1 | m | Less than 1% |
| Toluene | 8.9 | p | Less than 1% |
| Chloroform | 9.3 | p | 22-25% |
| Methylene Chloride | 9.7 | p | 19% |
| Carbon Tetrachloride | 8.6 | p | 2% |
| MMA | 8.8 | m | Less than 1% |
| 40% MMA-60% DMSO | 10.7 | calc. | 7.6% @ 60° C. |

Solubility parameter units are (cal/cm³)^{1/2}. Solubility parameter of PET is 10.7 (cal/cm³)^{1/2}. Notations beside solubility parameters are H-bonding groups and refer to the strength of hydrogen bonding by the material, where s = strongly, m = moderately and p = poorly bonded [Brandrup et al, eds., Polymer Handbook, 2nd edition, John Wiley & Sons, New York (1975)].

Based on the foregoing, the preferred solvents would be chlorinated hydrocarbons such as chloroform, methylene chloride and carbon tetrachloride. Other suitable solvents include 1,2-dichloroethane, isobutyl chloride and alkanes such as pentane, octane, isopentane, heptane and the like, as well as tetrahydrofuran, dimethyl sulfoxide, ethyl acetate, methylethylketone, pyridine, dimethyl formamide and the like.

Although any suitable siloxane monomer or oligomer may be employed in the practice of the invention, it is preferred to use dimethylsiloxane due to its ready availability, attractive cost factors and low degree of thrombogenicity possessed by polysiloxanes formed therefrom.

In a preferred embodiment, the substrate is pre-modified to enhance the attachment of the polysiloxane to the substrate by first exposing the substrate to a solvent solution of a vinyl-terminated polysiloxane, e.g., methacryloxypropyl terminated polydimethylsiloxane (MAOP-t-PDMS) or polymethoxyvinylsiloxane. Gamma-radiation induced free-radical graft polymerization of the MAOP-t-PDMS results in a substrate wherein a covalently grafted MAOP-t-PDMS is integrally and homogeneously distributed within and on the surface of the substrate. Presoaking the substrate in a solution of vinyl monomer, e.g., methyl methacrylate (MMA), prior to initiating polymerization results in enhanced graft polymerization. Subjecting this intermediate product to the above-described novel method results in covalent bonding between the polysiloxane coating and the MAOP-t-PDMS system, both on the surface of the substrate and in the interior thereof, thereby providing an even more stable coating which is less subject to delamination and/or degradation.

During the gamma radiation portion of the pre-treatment where MAOP-t-PDMS is used after a presoak in MMA, other polymers which are not vinyl functional may also be incorporated. Upon exposure to gamma radiation in the presence of the MMA monomer, these copolymers will become active/functional and bond and cross-link with the MMA and PET substrate. Examples of such polymers are: (acryloxypropyl)-methyl polydimethylsiloxane copolymer, (methyl methacryloxypropyl)polydimethylsiloxane copolymer, methacryloxypropyl polydimethylsiloxane copolymers, poly(methacryloxypropylmethyl)siloxane and poly(acryloxypropylmethyl)siloxane.

Whether the substrate is only coated or first subjected to pre-modification prior to coating, the resulting product has a greatly reduced thrombogenicity and high degree of adherence of the coating to the substrate, thereby rendering the structure highly stable.

The coating may be of any desired suitable thickness, e.g., from about 10 nanometers to about 1 mm, preferably from about 500 nanometers to about 5 μm.

The invention is illustrated by the following non-limiting examples wherein Dacron® refers to polyethyleneterephthalate (PET).

### EXAMPLE 1

Group A: Control Dacron®—Unmodified Dacron® Fabric (low porosity fabric)

Group B: MAOP-t-PDMS Pre-modified Dacron® (1):

Presoak Dacron® in a 10% solution of methyl methacrylate (MMA) monomer in 90% chloroform at room temperature for 24 hours. The presoak was followed by placing the sample into a solution of 10% MAOP-t-PDMS, 10% MMA, 80% chloroform, degassing the solution and exposing it to gamma radiation for a total dose of 0.10 to 0.15 Mrad. The samples were then washed in chloroform until no traces of monomer or unbonded polymer were detected.

Group C: MAOP-t-PDMS Pre-modified Dacron® (2):

Presoak Dacron® in a 40% solution of methyl methacrylate (MMA) and 60% dimethylsulfoxide (DMSO) for 24 hours. The presoak was followed by placing the sample into a solution of 10% MAOP-t-PDMS, 10% MMA, 80% chloroform, degassing the solution and exposing it to gamma radiation for a total dose of 0.10 to 0.15 Mrad. The samples were then washed in chloroform until no traces of monomer or unbonded polymer were detected.

Group E: Coated Dacron® (Dacron® which was soaked in a 10% PDMS-chloroform solution for 4 hours):

The PDMS in the solution was a two-part mixture of oligomers from Shincor Silicones (KE 1935 A and KE 1935 B) mixed in equal ratios. The sample was removed from the solution and cured thermally at 60° C. in air for 24 hours. The sample was then subjected to vacuum (30 in Hg) for 12 hours, soaked in chloroform for 12 hours and cured under vacuum again for 12 hours.

Concentrations higher than 10% provide thicker coatings. A solution concentration of 25% in hexane (rather than chloroform), for example, provides a coating on the order of 50 microns. The thickness of the coating can, therefore, be tailored to the specific application.

Group F: MAOP-t-PDMS Pre-modified Dacron® (1) followed by coating:

Group B process followed by Group E process.

Group G: MAOP-t-PDMS Pre-modified Dacron® (2) followed by coating:

Group C process followed by Group E process.

Group K: MAOP-t-PDMS Pre-modified Dacron® Control (3):

This process involves no presoaking. The process involves placing samples into a solution of 10% MAOP-t-PDMS, 10% MMA, 80% chloroform, degassing the solution and exposing it to gamma radiation for a total dose of 0.10 to 0.15 Mrad. The samples were then washed in chloroform until no traces of monomer or unbonded polymer were detected. Group K samples are not coated.

### EXAMPLE 2

Weight Increase (Weight Percent of PDMS Coating):

Gravimetric analysis of the modifications show various extents of weight increase (Table 2). The coated samples (Group E) show a weight increase of approximately 19% for the given conditions. Changes in solution concentration and dipping times will change the total weight increase. MAOP-t-PDMS pre-modified samples show varying weight increases depending on the technique used, with method 1 (Group B) having a 2-3% weight increase, method 2 (Group C) having a 4-5% weight increase, and method 3 (Group K) having less than a 1% weight increase. Changes in solution concentrations, presoaking times and irradiation doses will change these weight increases as more or less MAOP-t-PDMS, MMA and coated PDMS are incorporated.

TABLE 2

| Solution | Coating | Percent Mass | Coating Weight |
|---|---|---|---|
| Concentration | Time | Increase | (μg/mm²) |
| 5% PDMS-chloroform | 1 hour | 0.4 | 6.9 |
| 5% PDMS-chloroform | 4 hours | 6.9 | 9.5 |
| 5% PDMS-chloroform | 20 hours | 10.3 | 21.7 |
| 10% PDMS-chloroform | 1 hour | 13.6 | 21.1 |
| 10% PDMS-chloroform | 4 hours | 19.0 | 26.5 |
| 10% PDMS-chloroform | 20 hours | 22.3 | 17.8 |
| 20% PDMS-chloroform | 1 hour | 58.0 | 89 |
| 20% PDMS-chloroform | 4 hours | 42.0 | 60 |
| 20% PDMS-chloroform | 20 hours | 55.0 | 82 |

XPS Surface Chemistry Analysis:

X-ray photoelectron spectroscopy (XPS) was used to analyze the atomic concentration of the surfaces. Table 3 shows the atomic surface concentrations for Dacron®, coated Dacron®, MAOP-t-PDMS pre-modified Dacron®, MAOP-t-PDMS and coated Dacron®, and PDMS. From these data, it is clear that the surface coating following the method of this invention has the same composition as PDMS.

TABLE 3
CARBON, OXYGEN AND SILICON ATOMIC
CONCENTRATIONS FOR DACRON ® WITH COATINGS OF
PDMS AND GAMMA POLYMERIZED MAOP-t-PDMS AS
DETERMINED WITH XPS

| | Sample Modification | | | |
|---|---|---|---|---|
| Sample Set | Procedure | % Carbon | % Oxygen | % Silicon |
| Group A | Control Dacron ® - | 72.92 | 24.02 | 3.06 |
| | unmodified | | | |
| Group B | Pre-modified (1) | 53.41 | 21.88 | 24.71 |
| Group C | Pre-modified (2) | 61.10 | 21.78 | 17.12 |
| Group E | Coated | 49.44 | 22.45 | 28.11 |
| Group K | Pre-modified (3) | 67.40 | 24.50 | 8.10 |
| PDMS Film | Cured Silicone Film | 50.25 | 22.60 | 27.15 |

These data show that the pre-modification with MAOP-t-PDMS provides a bonded PDMS surface. Furthermore, following coating, the surface chemistry is comparable to silicone film, indicating complete coverage. Data for Group K modifications, pre-modifications with MAOP-t-PDMS, show bonding or attachment of a PDMS surface without presoaking in MMA. This indicates that the MAOP-t-PDMS pre-modification process is effective with or without the presoaking steps in MMA.

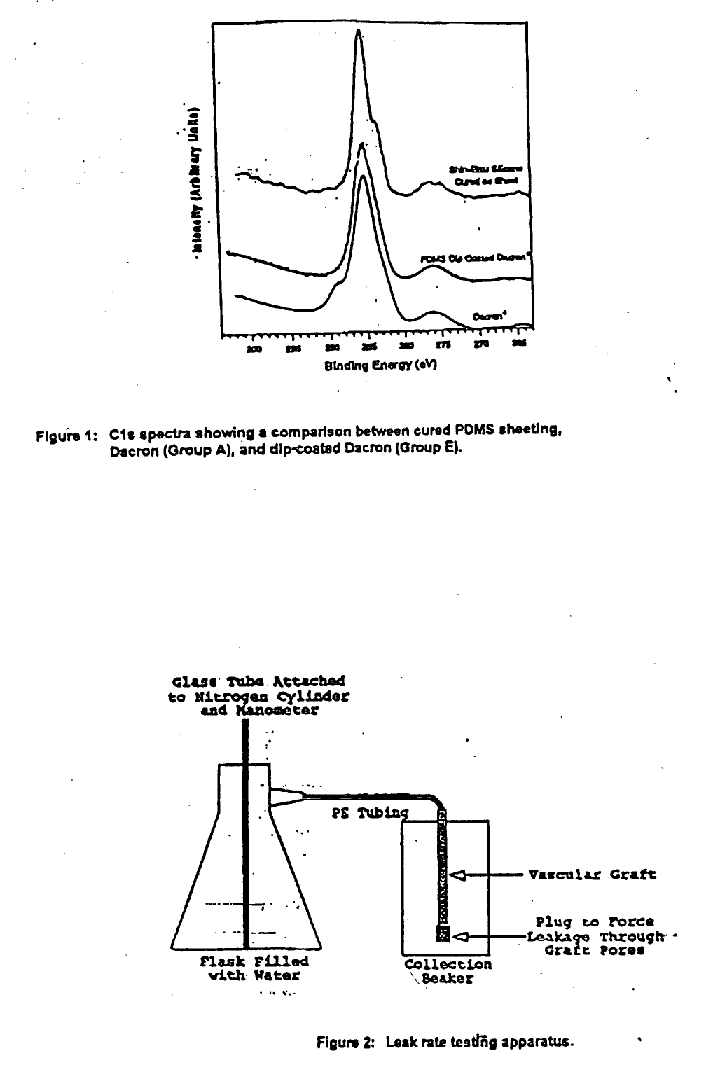

FIG. 1 shows the carbon (cls) XPS spectra for PDMS cured as a sheet, coated Dacron® (Group E) and Dacron® (Group A). The absence of the shoulder at 288 eV on the coated sample (Group E) compared to the unmodified Dacron® (Group A) indicates complete coverage of the Dacron® substrate. The absence of the shoulder at 283 eV on the coated sample (Group E) compared to the cured PDMS sheet indicates a more complete cure determined by the absence of residual vinyl (carbon-carbon) double bonds. The PDMS sheeting in this example was cured to the manufacturer's recommendations (150° C. in air for 1 hour).

Leak Rate:

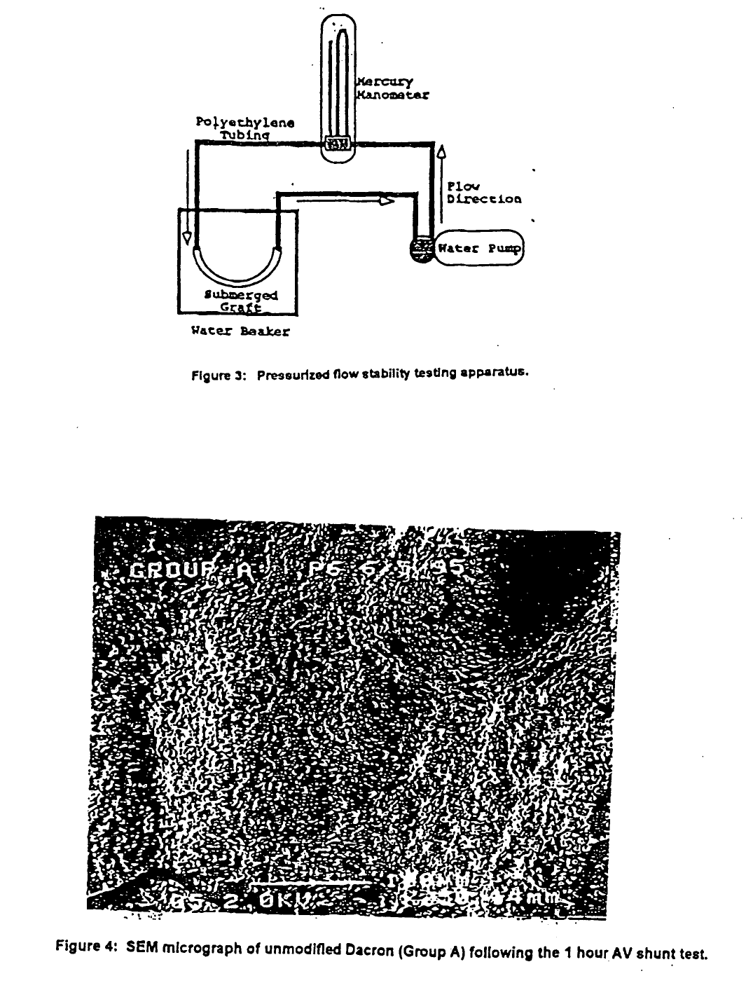

The coated samples may be rendered non-porous by the silicone coating on the substrate, if desired. The leak rate of water through the pores was evaluated using a pressurized flow system (FIG. 2). Grafts were placed in series with a pressure manometer and water reservoir and a back pressure of nitrogen. The volume of water leaking through the graft surface at 120 mm Hg in one minute was measured. The reported values for leaking are normalized for surface area and are reported in ml/cm²/min.

The leak rate for unmodified Dacron® was 320 ml/cm²/min. and the leak rate for a PDMS coated sample (4 hours in 10% PDMS-chloroform solution, followed by a 48-hour thermal cure at 60° C.) was 200 ml/cm²/min.

Coating Stability:

The coating stability was determined by attempting to wash or remove the surface coating under pressurized flowing conditions (FIG. 3). Dacron® prostheses were placed in a series flow system pressurized to 120 mm Hg with a surfactant solution (10% aqueous Triton X). The solution was allowed to flow through the lumen of each sample for 48 hours at a flow rate of 300 ml/min. The water flowing through the lumen and the solution which leaked from each prosthesis were collected and analyzed for the atomic concentration of elemental silicon with an inductively coupled plasma (ICP) using a Plasma 40 ICP from Perkin-Elmer. (Window size of 0.1 nm, a photo multiplier tube voltage of 700 V, and an integration of 680 msec—providing a detection limit of ±0.1 ppm, which corresponds to a concentration of ±0.1 μg/ml.) Table 4 shows ICP data for coated and control samples in two separate experiments.

TABLE 4
SILICON CONCENTRATIONS IN SOLUTION FROM THE
VASCULAR PROSTHESIS STABILITY STUDY
AS MEASURED BY ICP

| | Silicon | | Standard | |
|---|---|---|---|---|
| | Concentration | | Deviation | |
| Sample in Ultrapure ™ Water | | | | |
| 100 ppm Standard | 106.1 | ppm | 1.16 | ppm |
| Ultrapure Water | 0.4 | ppm | 0.04 | ppm |
| 12 hour flow with Dacron ® | 0.6 | ppm | 0.02 | ppm |
| 24 hour flow with Coated Dacron ® | 0.0 | ppm | 0.02 | ppm |
| Sample in 10% Triton X Solution | | | | |
| 100 ppm Silicon Standard | 101.6 | ppm | 0.59 | ppm |
| 10% Triton X Solution | 1.3 | ppm | 0.06 | ppm |
| No Prosthesis | | | | |
| 24 hour flow | | | | |
| 10% Triton X Solution | 1.6 | ppm | 0.07 | ppm |
| Coated Dacron ® | | | | |
| 48 hour flow | | | | |

These data indicate no silicone comes off the surface to the detection limit of the ICP used in this example, in a concentration greater than 0.1 μg/ml. This is indicative of the coating stability and no silicone will, therefore, be removed in vivo from the surface following implantation.

Reduced Thrombogenicity:

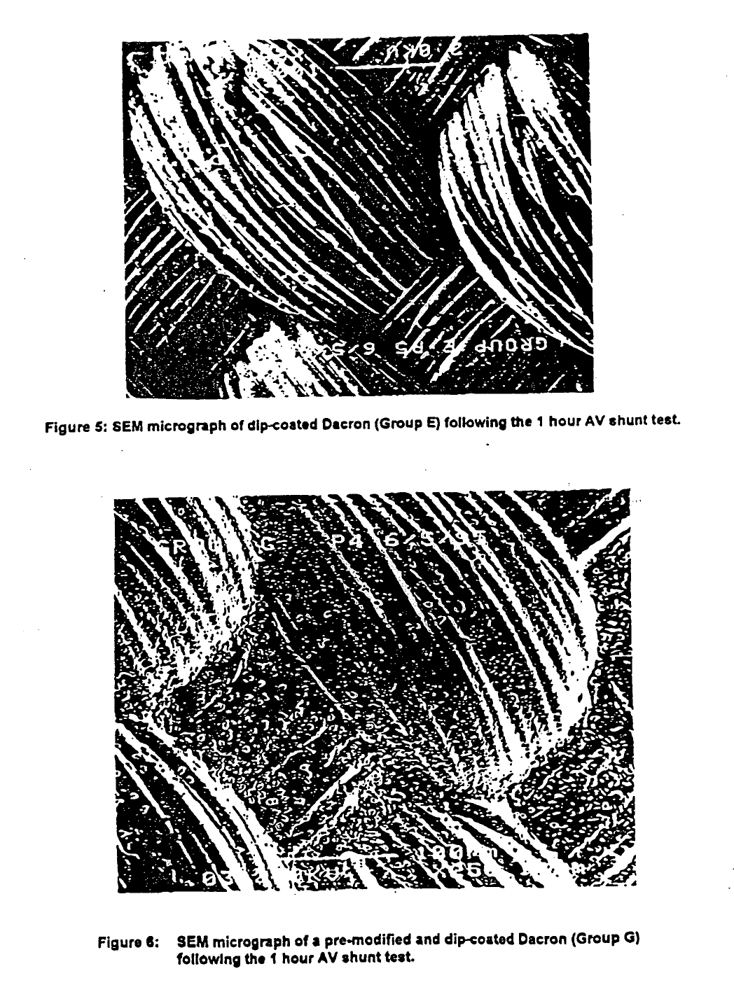

Using aseptic technique, arteriovenous (AV) shunts were constructed between the carotid artery and the jugular vein of adult mongrel canines. Samples to be tested were placed into a section of Silastic tubing and sealed in place with silicone RTV. Autologous ¹¹¹Indium labeled platelets were injected into the dogs and blood flow over the samples allowed for 60 minutes. The samples were then removed from the shunt and counted in a gamma-counter (Auto-logic, Abbott Laboratories). The counts shown in Table 5 are normalized to the surface area and are reported as counts/mm². SEM micrographs are shown in FIGS. 4-6 for these samples. Deposits are clearly visible, showing the distinct improvement achieved for the coated surfaces.

TABLE 5
MEAN PLATELET COUNTS FROM EX VIVO AV SHUNT
EXPERIMENTS FOR UNMODIFIED (GROUP A),
COATED (GROUP E) AND VARIOUS MAOP-t-PDMS
MODIFIED AND COATED (GROUPS B, C, F AND G)
DACRON ® SAMPLES

| Sample Set | Sample Modification Procedure | Mean Platelet Counts |
|---|---|---|
| Group A | Control Dacron ® - Unmodified | 2227.2 |
| Group B | Pre-modified (1) | 2035.8 |
| Group C | Pre-modified (2) | 2523.8 |
| Group D | Coated | 549.1 |
| Group E | Pre-modified (1) and Coated | 839.5 |
| Group F | Pre-modified (2) and Coated | 593.0 |

ANOVA confidence overlaps indicate no significant difference between groups A, B and C, or between Groups D, E and F, but indicates a significant difference between the two sets (A, B, C compared to coated samples D, E, F).

These data show an initial reduction in thrombogenicity with the coatings of PDMS with or without the pre-modification using MAOP-t-PDMS and MMA.

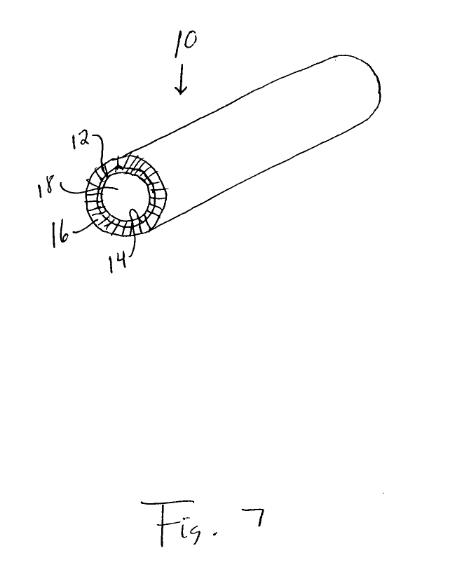

In the drawings, an embodiment of a blood-contacting device is set forth in FIG. 7 which is a schematic representation of a vascular prosthesis 10. The prosthesis has a generally tubular shape as defined by cylindrical member 12 having a luminal surface 14. The surface 14 is coated with the polymerized monomer or oligomer 16 which extends below surface 14 to a depth defined by the cross-hatchings in the figure. An axial luminal pathway 18 contained within the prosthesis provides a channel through which blood can flow.

1. A method of fabricating a device comprising a substrate having a surface of low thrombogenicity for contact with blood comprising the steps of:
(a) contacting said substrate with a solution of monomer or oligomer in a solvent to wet said surface and impregnate the substrate below said surface with said solution;
(b) removing said solvent to form a continuum of said monomer or oligomer on said surface and within said substrate; and
(c) polymerizing said monomer or oligomer to form a substantial continuum of polymer coating on said surface and extending within said substrate, thereby firmly anchoring said polymer coating on said surface.

2. The method of claim 1 wherein said monomer or oligomer is a siloxane monomer or oligomer and said polymer is a polysiloxane.

3. The method of claim 2 wherein said polysiloxane is a polyalkylsiloxane, a polyfluorosiloxane, a polyarylsiloxane, a polyalkyleneoxide siloxan, a copolymer or mixture thereof.

4. The method of claim 2 wherein said polysiloxane is polydimethylsiloxane.

5. The method of claim 1 wherein said device is a vascular prosthesis wherein the surface for contacting blood comprises the luminal surface thereof.

6. The method of claim 1 wherein said polymer has a lower degree of thrombogenicity than said surface of said substrate.

7. The method of claim 1 wherein said substrate of said device is constructed of a material selected from the group consisting of fibers, tubes, molded parts, expanded synthetic polymers, and woven, knitted, braided and non-woven fabrics.

8. The method of claim 7 wherein said synthetic polymer substrate is selected from the group comprising polyolefins, i.e., polypropylene, polyethylene, fluoropolymers, i.e., PTFE, PVF₂, polyesters, i.e., PET, PBT, and polyurethanes.

9. The method of claim 8 wherein said synthetic polymer is a fluorinated polyethylene.

10. The method of claim 8 wherein said synthetic polymer is polytetrafluoroethylene.

11. The method of claim 7 wherein said fabric is a polyethylene terephthalate fabric.

12. The method of claim 1 wherein said solvent is an organic, volatile solvent and is removed in step (b) by evaporation.

13. The method of claim 12 wherein said solvent is a chlorinated hydrocarbon, an alkane, tetrahydrofuran, dimethylsulfoxide, a cycloalkane, an alkylbenzene, an alkyl acetate, a ketone, pyridine or dimethylformamide.

14. The method of claim 5 wherein said vascular prosthesis is a small diameter vascular prosthesis.

15. The method of claim 1 wherein the thickness of the polymer coating on the surface is from about 10 nanometers to about 1 mm.

16. The method of claim 1 wherein said polymerization is effected at an elevated temperature.

17. The method of claim 2 including, prior to step (a), the steps:
(i) contacting said substrate of said device with a solution of a vinyl-terminated polysiloxane capable of graft polymerization onto said substrate to wet said surface and impregnate the substrate below said surface to a substantial depth with said solution; and
(ii) graft polymerizing said vinyl-terminated polysiloxane onto said surface and within said substrate.

18. The method of claim 17 wherein said vinyl-terminated polysiloxane is acryloxypropyl- or methacryloxypropyl-terminated polydimethylsiloxane or polymethoxyvinylsiloxane.

19. The method of claim 17 wherein said graft polymerization is a gamma-radiation induced free radical polymerization.

20. The method of claim 17 wherein, prior to step (i), said substrate of said device is presoaked in said terminal vinyl monomer or a solution thereof.

21. The device produced by the method of claim 1.

22. The device produced by the method of claim 17.

23. A device comprising a substrate having a surface for contacting blood, said surface comprising a substantial continuum of thrombus-resistant polymeric material coated on and extending into said substrate.

24. The device according to claim 23 comprising a vascular prosthesis wherein the surface for contacting blood comprises the luminal surface thereof.

25. The vascular prosthesis of claim 24 comprising an elongated tubular segment open at both ends, wherein the surface for contacting blood comprises the luminal surface defining a confined-flow passageway through said tubular segment.

26. The device of claim 23 wherein said polymeric material is a polysiloxane.
